(19) United States
(12) Patent Application Publication
(10) Pub. No.: US 2008/0213454 A1
(43) Pub. Date: Sep. 4, 2008

(54) Genetic transformation of apple without use of a selectable marker

(76) Inventors: Herb S. Aldwinckle, Geneva, NY (US); Mickael A. Malnoy, Mezzolombardo (IT); Ewa E. Borejsza-Wysocka, Geneva, NY (US)

Correspondence Address: CLARK & ELBING LLP, 101 FEDERAL STREET, BOSTON, MA, 02110, US

(21) Appl. No.: 11/973,539

(22) Filed: Oct. 9, 2007

Related U.S. Application Data
(60) Provisional application No. 60/849,881, filed on Oct. 9, 2006.

(51) Int. Cl. A23L 2/02; A01H 5/00; A23L 1/48; C12N 15/11

(52) U.S. Cl. 426/599; 800/315; 800/300; 800/301; 800/278; 426/531

(57) ABSTRACT

Disclosed are apple plants or apple plant components that include an isolated polynucleotide free from a selectable marker polynucleotide. Methods of producing such plants are also disclosed.

### CROSS REFERENCE TO RELATED APPLICATIONS

This application claims benefit of U.S. provisional application Ser. No. 60/849,881, filed Oct. 9, 2006, the disclosure of which is hereby incorporated by reference in its entirety.

### STATEMENT AS TO FEDERALLY FUNDED RESEARCH

This invention was made, in part, with funding from the United States Department of Agriculture-Cooperative State Research Education, and Extension Service, and a grant from the Binational Agricultural Research and Development Fund. The U.S. Government may have certain rights in the invention.

### BACKGROUND OF THE INVENTION

The invention relates to transformation of apple plants.

Selectable marker genes are widely used for the efficient transformation of crop plants. In most cases, selection is based on antibiotic or herbicide resistance. These marker genes are preferred because they tend to be most efficient (for example, in apple a 2-20% transformation rate is generally achieved and, at times, an 80% transformation rate may be achieved using the nptII gene for kanamycin resistance). Due mainly to consumer and grower concerns, considerable effort is being expended to develop a suite of strategies (for example, site-specific recombination, homologous recombination, transposition, and co-transformation) to eliminate the marker gene from the nuclear or chloroplast genome after selection (Miki and McHugh, 2004). Current efforts concentrate on systems where the marker genes are eliminated efficiently soon after transformation. However, these methods are tedious and of doubtful reliability. Alternatively, transgenic plants can be produced using marker genes that do not rely on antibiotic or herbicide resistance but instead promote regeneration after transformation. Examples of such transformation methodologies include the use of phosphomannose isomerase (Joersbo et al., 1998), xylulose isomerase (Haldrup et al., 1998a; b), β-glucuronidase (Joersbo and Okkels, 1996), or isopentyl transferase (Endo et al., 2001). These methods, however, are generally of much lower efficiency than use of nptII.

For the commercialization of transgenic plants use of a completely marker-free technology is preferable, since there would be no involvement of antibiotic resistance genes or other marker genes with negative connotations. This would eliminate the marker gene as a source of consumer and activist concern, and would simplify the regulatory process. It would also facilitate a second cycle of transformation of selected transformed lines. With this goal in mind, this application presents methods for apple transformation without the use of a selectable marker. By implementing this technique, markerless apple plant tissue is routinely generated.

### SUMMARY OF THE INVENTION

We have developed a technique for apple transformation without use of a selectable marker, generating so-called markerless apple plants.

Apple plant tissue disclosed herein is free of a selectable (screenable) marker. Such markers include a gene that, if expressed in apple plants or apple plant tissues, makes it possible to distinguish them from other apple plants or apple plant tissues that do not express that gene. Exemplary selectable markers include genes encoding resistance to an antibiotic, herbicide or toxic compound can be used to identify transformation events. The present invention, in general, provides transgenic apple plants free of selectable markers such as antibiotic resistance genes (such as streptomycin phosphotransferase (SPT) (a gene encoding streptomycin resistance), neomycin phosphotransferase (NPTII) (a gene encoding kanamycin and geneticin resistance), or other similar genes known in the art); or herbicide resistance genes (such as hygromycin phosphotransferase (HPT or APHIV) (a gene encoding resistance to hygromycin), acetolactate synthase (als) (genes encoding resistance to sulfonylurea-type herbicides), or genes (BAR and/or PAT) coding for resistance to herbicides which act to inhibit the action of glutamine synthase such as phosphinothricin (Liberty or Basta), or other similar genes known in the art.)

In one aspect, the invention features an apple plant or plant component comprising an isolated polynucleotide free from a selectable marker polynucleotide. In preferred embodiments, the apple plant or plant component is free from a selectable marker polynucleotide encoding an antibiotic resistance gene. In still other preferred embodiments, the apple plant or plant component is free from an herbicide tolerance protein. Preferably, the isolated polynucleotide encodes a disease resistance gene, such as MpNPR1, which provides resistance to a pathogen. Exemplary apple plant components include apple fruit, apple seed, an apple fruiting scion, an apple rootstock, an apple interstem, apple pollen, or apple regenerable tissue.

In preferred embodiments, the apple plant or apple plant component expresses the isolated polynucleotide. In general, by “express” or “expression” means the combination of intracellular processes, including transcription and translation, undergone by a coding DNA molecule such as a structural gene to produce a polypeptide or an RNA molecule, without concomitant polypeptide production.

In other aspects, the invention features a beverage, a food product such as fresh fruit, or dietary fiber produced using any of the transgenic apple plants or apple plant components described herein.

In still another aspect, the invention features a method of transforming an apple plant cell or apple plant tissue using an Agrobacterium-mediated process including the steps of: a) inoculating said apple plant cell or apple plant tissue with Agrobacterium containing at least one genetic component or trait capable of being transferred to the apple plant cell or apple tissue in an inoculation media, the inoculation media being free from a selectable marker; and b) selecting transformed apple plant cells or apple plant tissue.

In preferred embodiments, the method further includes regenerating a transgenic apple plant expressing the genetic component or trait capable of being transferred to the apple plant cell or apple tissue from the selected transformed apple plant cells or apple tissue.

According to the methods of the present invention, transformation rates between 5-10%, 10-15%, 15-20%, 20-25%, and up to 30% may be typically achieved to produce transgenic apple plants or transgenic apple components that include an isolated polynucleotide free of a selectable marker.

Regeneration of a transgenic apple plant from a transformed apple cell is the process of growing a plant from a plant cell (e.g., plant protoplast or explant).

A transformed cell is typically a cell whose DNA has been altered by the introduction of an exogenous DNA molecule into that cell.

Transformation, in general, may occur under natural or artificial conditions using various methods well known in the art. Such transformation may rely on any known method for the insertion of nucleic acid sequences into an apple host cell, including Agrobacterium-mediated transformation protocols, viral infection, whiskers, electroporation, microinjection, polyethylene glycol-treatment, heat shock, and particle bombardment.

A transgenic cell refers to any cell derived or regenerated from a transformed cell or derived from a transgenic organism. Exemplary transgenic cells include plant calli derived from a transformed plant cell and particular cells, such as somatic cells (e.g., leaf, root, stem) or reproductive (germ) cells, obtained from a transgenic plant.

A “transgenic plant” is a plant or progeny thereof derived from a transformed plant cell or protoplast, wherein the plant DNA contains an introduced exogenous DNA molecule not originally present in a native, non-transgenic plant of the same strain. The terms “transgenic plant” and “transformed plant” have sometimes been used in the art as synonymous terms to define a plant whose DNA contains an exogenous DNA molecule. However, it is thought more scientifically correct to refer to a regenerated plant or callus obtained from a transformed plant cell or protoplast as being a transgenic plant and that usage will be followed herein.

“Plant” is used herein in a broad sense and refers to differentiated plants as well as undifferentiated plant material, such as protoplasts, plant cells, seeds, plantlets, etc., that under appropriate conditions can develop into mature plants, the progeny thereof, and parts thereof such as cuttings and fruits of such plants.

A “plant cell” refers to any self-propagating cell bounded by a semi-permeable membrane and typically is one containing a plastid. Such a cell also typically requires a cell wall if further propagation is desired. Plant cell, as used herein includes, without limitation, seeds, suspension cultures, embryos, meristematic regions, callus tissue, leaves, roots, shoots, gametophytes, sporophytes, pollen, and microspores.

A “plant component” refers to a part, segment, or organ obtained from an intact plant or plant cell. Exemplary plant components include, without limitation, somatic embryos, leaves, stems, roots, flowers, fruits, scions, and rootstocks.

A “pathogen” refers to an organism whose infection of viable plant tissue elicits a disease response in the plant tissue. Such pathogens include, without limitation, bacteria, mycoplasmas, fungi, insects, nematodes, viruses, and viroids. Exemplary apple diseases include apple fire blight, apple scab and apple rust diseases, and apple powdery mildew disease.

Other features and advantages of the invention will be apparent from the following Detailed Description, the drawings, and the claims.

### BRIEF DESCRIPTION OF THE DRAWINGS

FIG. 1 shows a β-glucuronidase (GUS) histochemical test on microshoots of M.26, M.26 pwiATT 35SGus nptII and putative transgenic M.26 pwiATT 35SGus.

### DETAILED DESCRIPTION OF THE INVENTION

The materials and methods provided herein can be used to make apple plants, apple plant components, and apple plant products free of a selectable marker. For example, apple plants free of an undesirable selectable trait (such as an antibiotic resistance gene or an herbicide resistant gene). The methods, in general, include transforming an apple plant cell with one or more nucleic acid molecules that encode a polypeptide or an RNA molecule, wherein expression of the one or more polypeptides or one or more RNAs results in modulated levels (e.g., increased or decreased levels) of one or more desirable traits as discussed herein. Apple plants and apple plant components produced using such methods can be used according to the methods in the art, for example, as food sources for sugars.

### Polynucleotides

Isolated nucleic acid molecules useful in the invention are known in the art, including isolated nucleic acid molecules that encode any number of desirable traits in an apple plant or may be introduced into an apple plant. The terms “nucleic acid molecule” and “polynucleotide” are used interchangeably herein, and refer to both RNA and DNA, including cDNA, genomic DNA, synthetic (e.g., chemically synthesized) DNA, and DNA (or RNA) containing nucleic acid analogs. Polynucleotides can have any three-dimensional structure. A nucleic acid molecule can be double-stranded or single-stranded (i.e., a sense strand or an antisense strand). Non-limiting examples of polynucleotides include genes, gene fragments, exons, introns, messenger RNA (mRNA), transfer RNA, ribosomal RNA, siRNA, micro-RNA, ribozymes, cDNA, recombinant polynucleotides, branched polynucleotides, plasmids, vectors, isolated DNA of any sequence, isolated RNA of any sequence, nucleic acid probes, and primers, as well as nucleic acid analogs.

As used herein, the term “isolated,” when in reference to a nucleic acid molecule, refers to a nucleic acid molecule that is separated from other nucleic acid molecules that are present in a genome, e.g., an apple plant genome, including nucleic acid molecules that normally flank one or both sides of the nucleic acid molecule in the apple genome. The term “isolated” as used herein with respect to nucleic acid molecules also includes any non-naturally-occurring sequence, since such non-naturally-occurring sequences are not found in nature and do not have immediately contiguous sequences in a naturally-occurring genome.

An isolated nucleic acid molecule can be, for example, a DNA molecule, provided one of the nucleic acid sequences normally found immediately flanking that DNA molecule in a naturally-occurring genome is removed or absent. Thus, an isolated nucleic acid molecule includes, without limitation, a DNA molecule that exists as a separate molecule (e.g., a chemically synthesized nucleic acid molecule, or a cDNA or genomic DNA fragment produced by the polymerase chain reaction (PCR) or restriction endonuclease treatment) independent of other sequences, as well as DNA that is incorporated into a vector, an autonomously replicating plasmid, a virus (e.g., pararetrovirus, retrovirus, lentivirus, adenovirus, adeno-associated virus, or herpesvirus), or the purified genomic DNA of a prokaryote or eukaryote. In addition, an isolated nucleic acid molecule can include an engineered nucleic acid molecule such as a DNA molecule that is part of a hybrid or fusion nucleic acid molecule.

A nucleic acid molecule can be made, for example, by chemical synthesis or using PCR. PCR refers to a procedure or technique in which target nucleic acid molecules are amplified. PCR can be used to amplify specific sequences from DNA as well as RNA, including sequences from total genomic DNA or total cellular RNA. Various PCR methods are described, for example, in PCR Primer: A Laboratory Manual, Dieffenbach and Dveksler, eds., Cold Spring Harbor Laboratory Press, 1995. Generally, sequence information from the ends of the region of interest or beyond is employed to design oligonucleotide primers that are identical or similar in sequence to opposite strands of the template to be amplified. Various PCR strategies also are available by which site-specific nucleotide sequence modifications can be introduced into a template nucleic acid molecule.

An isolated nucleic acid molecule may also include the regulatory regions which control expression of a polypeptide encoded by the isolated nucleic acid molecule.

The term “exogenous” with respect to a nucleic acid molecule indicates that the nucleic acid molecule is part of a recombinant nucleic acid molecule construct, or is not in its natural environment. For example, an exogenous nucleic acid molecule can be a sequence from one species introduced into another species, i.e., a heterologous nucleic acid molecule. Typically, such an exogenous nucleic acid molecule is introduced into the other species via a recombinant nucleic acid molecule construct. An exogenous nucleic acid molecule can also be a sequence that is native to an organism and that has been reintroduced into cells of that organism. An exogenous nucleic acid molecule that includes a native sequence can often be distinguished from the naturally occurring sequence by the presence of non-natural sequences linked to the exogenous nucleic acid molecule, e.g., non-native regulatory regions flanking a native sequence in a recombinant nucleic acid molecule construct. In addition, stably transformed exogenous nucleic acid molecules typically are integrated at positions other than the position where the native sequence is found. It will be appreciated that an exogenous nucleic acid molecule may have been introduced into a progenitor and not into the cell under consideration. For example, a transgenic apple plant containing an exogenous nucleic acid molecule can be the progeny of a cross between a stably transformed plant and a non-transgenic plant. Such progeny are considered to contain the exogenous nucleic acid molecule.

Any number of polynucleotides or nucleic acid molecules encoding one or more desirable traits are useful in the invention.

A “trait,” as used herein, is a distinguishing feature or characteristic of an apple plant, which may be altered according to the present invention by integrating one or more “polynucleotides” into the genome of at least one apple plant cell of a transformed apple plant. The polynucleotide(s) confer a change in the trait of a transformed apple plant, by modifying any one of a number of genetic, molecular, biochemical, physiological, morphological, or agronomic characteristics or properties of the transformed apple plant cell or apple plant as a whole. Thus, for example, expression of one or more, stably integrated desired polynucleotide(s) in an apple plant genome, may alter a trait that is selected from the group consisting of, but not limited to, increased drought tolerance, enhanced cold and frost tolerance, improved vigor, enhanced color, enhanced health and nutritional characteristics, improved storage, enhanced yield, enhanced salt tolerance, enhanced heavy metal tolerance, increased disease tolerance (for example, resistance to microbial (e.g., bacterial pathogens), fungal and viral pathogens, nematodes, as wells as insect pathogens), increased pest tolerance, increased water-stress tolerance, enhanced sweetness, improved vigor, improved taste, improved texture, resistance to flesh browning, enhanced columnar tree form, increased micronutrient uptake, improved starch composition, and improved longevity.

Methodologies to determine plant growth or response to stress include for example, height measurements, weight measurements, leaf area, ability to flower, water use, transpiration rates and yield. In some instances, a trait is visible to the human eye, such as seed or plant size, or can be measured by biochemical techniques, such as detecting the protein, starch or oil content of seed or leaves, or by the observation of the expression level of a gene or genes, e.g., by employing Northern analysis, reverse-transcription polymerase chain reaction (RT-PCR), real time PCR, microarray gene expression assays or reporter gene expression systems, or by agricultural observations such as stress tolerance, yield or pathogen tolerance. Any technique can be used to measure the amount of, comparative level of, or difference in any selected chemical compound or macromolecule in the transgenic apple plants.

The present invention therefore also includes trait modification of an apple plant. By “trait modification” is meant a detectable difference in a characteristic in an apple plant ectopically expressing a polynucleotide or polypeptide relative to a plant not doing so, such as a wild type plant. In some cases, the trait modification can be evaluated quantitatively. For example, the trait modification can entail at least about a 2% increase or decrease in an observed trait (difference), at least a 5% difference, at least about a 10% difference, at least about a 20% difference, at least about a 30%, at least about a 50%, at least about a 70%, or at least about a 100%, or an even greater difference. It is known that there can be a natural variation in the modified trait. Therefore, the trait modification observed entails a change of the normal distribution of the trait in the plants compared with the distribution observed in wild type plant.

Exemplary traits amendable to trait modification include those to seed (such as embryo or endosperm), fruit, root, flower, leaf, stem, shoot, seedling or the like, including: enhanced tolerance to environmental conditions including freezing, chilling, heat, drought, water saturation, radiation and ozone; improved tolerance to microbial (e.g., bacterial), fungal or viral diseases; improved tolerance to pest infestations, including nematodes; improved tolerance of heavy metals or enhanced ability to take up useful heavy metals such as iron; improved growth under poor photoconditions (e.g., low light and/or short day length), or changes in expression levels of genes of interest. Other phenotypes that can be modified relate to the production of plant metabolites, such as variations in the production of sterols, phytosterols, vitamins, wax monomers, anti-oxidants, amino acids, lignins, cellulose, tannins, phenyl lipids (such as chlorophylls and carotenoids), glucosinolates, or modified sugar (insoluble or soluble) and/or starch composition. Physical plant characteristics that can be modified include cell development, fruit and seed size and number, yields of plant parts such as stems, leaves and roots, the stability of the seeds during storage, characteristics of the seed pod (e.g., susceptibility to shattering), root hair length and quantity, internode distances, or the quality of seed coat. Plant growth characteristics that can be modified include growth rate, germination rate of seeds, vigor of plants and seedlings, leaf and flower senescence, male sterility, apomixis, flowering time, flower abscission, rate of nitrogen uptake, biomass or transpiration characteristics, as well as plant architecture characteristics such as apical dominance, branching patterns, number of organs, organ identity, organ shape or size.

According to the present invention, a polynucleotide also may be used to alter a trait associated with an apple plant. Such a polynucleotide, under such circumstances, may be used for pharmaceutical purposes, to express in plants a product of pharmaceutical relevance or importance. Examples of pharmaceutically relevant desired polynucleotides include, without limitation, those that encode peptides, nutraceuticals, antioxidants such as quercetin, vaccines, growth factors, and enzymes. Additional polynucleotides include a gene which modifies production of a secondary compound such as a flavonoid.

One or more polynucleotides or nucleic acid molecules that encode a trait can be used to transform an apple plant cell such that an apple plant produced from the apple plant cell has a modified trait. For example, a nucleic acid molecule encoding a disease resistance polypeptide can be used to transform an apple plant cell. A nucleic acid molecule encoding a lytic peptide can also be used to transform a plant cell.

Two or more polynucleotides or nucleic acid molecules that encode a trait can also be used to transform a plant cell such that a plant produced from the plant cell has a modified trait (e.g., increased disease resistance). For example, a first nucleic acid molecule encoding a polypeptide that includes an amino acid sequence corresponding to a MPNPR1 gene, and a second nucleic acid molecule encoding a polypeptide that includes an attacin E polypeptide can be used to transform an apple plant cell.

One skilled in the art recognizes that in certain instances that virtually any polypeptide coding for a trait may be expressed in an apple plant cell. The term “polypeptide” as used herein refers to a compound of two or more subunit amino acids, amino acid analogs, or other peptidomimetics, regardless of post-translational modification (e.g., phosphorylation or glycosylation). The subunits may be linked by peptide bonds or other bonds such as, for example, ester or ether bonds. The term “amino acid” refers to natural and/or unnatural or synthetic amino acids, including D/L optical isomers. Full-length proteins, analogs, mutants, and fragments thereof are encompassed by this definition.

By “isolated” or “purified” with respect to a polypeptide it is meant that the polypeptide is separated to some extent from the cellular components with which it is normally found in nature (e.g., other polypeptides, lipids, carbohydrates, and nucleic acid molecules). A purified polypeptide can yield a single major band on a non-reducing polyacrylamide gel. A purified polypeptide can be at least about 75 percent pure (e.g., at least 80 percent, 85 percent, 90 percent, 95 percent, 97 percent, 98 percent, 99 percent, or 100 percent pure). Purified polypeptides can be obtained by, for example, extraction from a natural source, by chemical synthesis, or by recombinant production in a host cell or transgenic plant, and can be purified using, for example, affinity chromatography, immunoprecipitation, size exclusion chromatography, and ion exchange chromatography. The extent of purification can be measured using any appropriate method, including, without limitation, column chromatography, polyacrylamide gel electrophoresis, or high-performance liquid chromatography.

One of skill in the art will further appreciate that for select purposes a polynucleotide expresses a nucleic acid molecule useful in silencing the expression of a gene.

Gene silencing is generally used to refer to suppression of expression of a gene. The degree of reduction may be so as to totally abolish production of the encoded gene product, but more usually the abolition of expression is partial, with some degree of expression remaining. The term should not therefore be taken to require complete “silencing” of expression. It is used herein where convenient because those skilled in the art well understand this phrase.

Gene silencing may be achieved using any standard method known in the art including RNAi and sense suppression technologies.

Suitable target genes for silencing will occur to those skilled in the art as appropriate to the problem in hand. For instance, in plants, it may be desirable to silence genes conferring unwanted traits in the plant by transformation with transgene constructs containing elements of these genes. Examples of this type of application include silencing of ripening specific genes in apple to improve processing and handling characteristics of the harvested fruit; silencing of genes involved in regulatory pathways controlling development or environmental responses to produce plants with novel growth habit or (for example) disease resistance; elimination of toxic secondary metabolites by silencing of genes required for toxin production. In addition, silencing can be useful as a means of developing virus resistant plants when the transgene is similar to a viral genome. Methods for silencing plant gene expression are known in the art, as well as evaluating the effects of such silencing on a plant such as apple.

### Recombinant Constructs and Vectors

Vectors containing nucleic acid molecules such as those described herein also are provided. A “vector” is a replicon, such as a plasmid, phage, or cosmid, into which another DNA segment may be inserted so as to bring about the replication of the inserted segment. Generally, a vector is capable of replication when associated with the proper regulatory regions. Suitable vector backbones include, for example, those routinely used in the art such as plasmids, viruses, artificial chromosomes, BACs, YACs, or PACs. The term “vector” includes cloning and expression vectors, as well as viral vectors and integrating vectors. An “expression vector” is a vector that includes one or more regulatory regions. Suitable expression vectors include, without limitation, plasmids and viral vectors derived from, for example, bacteriophage, baculoviruses, tobacco mosaic virus, herpesviruses, cytomegalovirus, vaccinia viruses, adenoviruses, adeno-associated viruses, and retroviruses. Numerous vectors and expression systems are commercially available from commercial sources such as Invitrogen/Life Technologies (Carlsbad, Calif.).

The term “regulatory region” refers to nucleotide sequences that influence transcription or translation initiation and rate, and stability and/or mobility of the transcript or polypeptide product. Regulatory regions include, without limitation, promoter sequences, enhancer sequences, response elements, protein recognition sites, inducible elements, promoter control elements, protein binding sequences, 5′ and 3′ untranslated regions (UTRs), transcriptional start sites, termination sequences, polyadenylation sequences, introns, translational enhancers (such as an enhancer obtained from an alfalfa mosaic virus) and other regulatory regions that can reside within coding sequences, such as secretory signals and protease cleavage sites. Such regulatory elements are well known in the art. It will be understood that more than one regulatory region may be present in a recombinant polynucleotide, e.g., introns, enhancers, upstream activation regions, and inducible elements. Thus, more than one regulatory region can be operably linked to the sequence encoding a sugar-modulating polypeptide. By “operably linked” is meant positioning of a regulatory region and a transcribable sequence in a nucleic acid molecule so as to allow or facilitate transcription of the transcribable sequence. For example, a regulatory region is operably linked to a coding sequence when RNA polymerase is able to transcribe the coding sequence into mRNA, which then can be translated into a protein encoded by the coding sequence.

The recombinant constructs disclosed herein typically include a polynucleotide sequence (e.g., a sequence encoding a sugar-modulating polypeptide) inserted into a vector suitable for transformation of plant cells. Recombinant vectors can be made using, for example, standard recombinant DNA techniques (see, e.g., Sambrook et al. (1989) Molecular Cloning: A Laboratory Manual, 2nd ed., Cold Spring Harbor Laboratory, Cold Spring Harbor, N.Y.).

### Transgenic Apple Plants and Cells

Any vector known in the art can be used to transform apple plant cells and, if desired, generate transgenic apple plants. Thus, transgenic apple plants and apple plant cells containing the nucleic acid molecules described herein also are provided, as are methods for making such transgenic apple plants and apple plant cells. An apple plant or apple plant cells can be transformed by having the construct integrated into its genome, i.e., can be stably transformed. Stably transformed cells typically retain the introduced nucleic acid molecule sequence with each cell division. Alternatively, the plant or plant cells also can be transiently transformed such that the construct is not integrated into its genome. Both transiently transformed and stably transformed transgenic apple plants and apple plant cells can be useful in the methods described herein.

Typically, transgenic apple plant cells used in the methods described herein constitute part or all of a whole plant. Such plants can be grown in a manner known in the art, for example, either in a growth chamber, a greenhouse, or in a field or orchard. Transgenic apple plants can be also be bred as desired for a particular purpose, e.g., to introduce a recombinant nucleic acid molecule into other lines, to transfer a recombinant nucleic acid molecule to other species, or for further selection of other desirable traits. Alternatively, transgenic apple plants can be propagated according to standard methods known in the art. Progeny includes descendants of a particular apple plant or an apple plant line.

Alternatively, transgenic apple plant cells can be grown in suspension culture, or tissue or organ culture, for production of secondary metabolites. For the purposes of the methods provided herein, solid and/or liquid tissue culture techniques can be used. When using solid medium, transgenic apple plant cells can be placed directly onto the medium or can be placed onto a filter film that is then placed in contact with the medium. When using liquid medium, transgenic apple plant cells can be placed onto a flotation device, e.g., a porous membrane that contacts the liquid medium. Solid medium typically is made from liquid medium by adding agar. For example, a solid medium can be Murashige and Skoog (MS) medium containing agar and a suitable concentration of an auxin, e.g., 2,4-dichlorophenoxyacetic acid (2,4-D), and a suitable concentration of a cytokinin, e.g., kinetin.

Techniques for transforming an apple plant cell are known in the art. The polynucleotides and/or recombinant vectors described herein can be introduced into the genome of a plant host using any of a number of known methods, including electroporation, microinjection, and biolistic methods. Preferably, such polynucleotides or vectors can be combined with suitable T-DNA flanking regions and introduced into a conventional Agrobacterium tumefaciens host vector. Such Agrobacterium tumefaciens-mediated transformation techniques, including disarming and use of binary vectors, are well known in the art. Other gene transfer and transformation techniques include protoplast transformation through calcium or PEG, electroporation-mediated uptake of naked DNA, electroporation of plant tissues, viral vector-mediated transformation, and microprojectile bombardment. If a cell or tissue culture is used as the recipient tissue for transformation, plants can be regenerated from transformed cultures using techniques known to those skilled in the art.

The polynucleotides and vectors described herein can be used to transform a number of apple cultivars. Exemplary cultivars useful in the invention, without limitation, include: Adina, Akane, Anna, Antonovka, Arkansas Black, Bancroft, Beacon, Beaujade, Belle de Boskoop, Big Time, Blushing Golden, Braeburn, Bramley's Seedling, Britegold, Cameo, Champion, Chenango, Chieftain, Cleopatra, Connel Red, Coromandel Red, Cortland, Cox's Orange Pippin, Crispin, Criterion, Dayton, Delicious (including Red Delicious), Democrat, Discovery, Dorsett Golden, Dulcet, Earliblaze, Earlidel, Earligold, Early Cortland, Ein Shemer, Elstar, Empire, Empress, Frameuse, Fiesta, Florina, Fortune, Freedom, Fuji, Gala, Galaxy, Geneva Early, Gingergold, Gloster, Golden Russet, Golden Delicious, Golden Supreme, Granny Smith, Gravenstein, Greensleeves, Grimes Golden, Haralson, Hauguan, Haushuai, Honeycrisp, Honeygold, Hatsuaki, Himekami, Hokuto, Idared, Iwakami, James Grieve, Jazz, Jerseymac, Jonafree, Jonagold, Jonagored, Jonalicious, Jonamac, Jonared, Jonasty, Jonathan, Jonnee, Jored, Karmijn, Kitakami, Laxton's Superb, Liberty, Lodi, Lurared, Lysgolden, Macoun, Maigold, McShay, McIntosh, Melrose, Mollies Delicious, Monroe, Mutsu, Northern Spy, Northwestern Greening, Nova Easygro, Novamac, Orin, Ozark Gold, Paulared, Pink Lady, Pinova, Pinata, Prima, Prime Gold, Primicia, Princessa, Priscilla, PureGold, Ralls Janet, Raritan, Red Baron, Redchief, Regent, Reine des Reinettes, Reinette du Canada, R.I. Greening, Rome Beauty, Rubinette, Sansa, Sayaka, Sekai-ichi, Senshu, Shamrock, Shizuka, Sir Prize, Smoothee, Spartan, Stayman, Winesap, Spigold, Splendor, State Fair, Sturmer Pippin, Summerdel, SummerRed, Summer Treat, Sundowner, Sunrise, Sweet Sixteen, Takana, Tompkins King, Tsugaru, Twenty Ounce, Tolman Sweet, Tydeman's Early Worcester, Viking, Vista Bella, Wealthy, Williams Pride, Winesap, Winter Banana, Wolf River, Worcester Pearmain, Yataka, Yellow Newtown, Yoko, York Imperial, 2085, and other Gala X Splendor clones. Each of these cultivars and all of their sports are useful in the invention.

Suitable apple rootstocks, without limitation, include M.7, M.9, M.26, M.27, MM. 106, MM.111, Merton 793, Maruba kaido, Budagovsky 9, Mark, Ottawa 3, and seedling (i.e., a rootstock propagated from a seed of known or unknown parentage).

A transformed apple plant cell, apple callus, apple tissue, or apple plant can be identified and isolated by selecting or screening the engineered plant material as described herein, for example, for particular traits. Such screening and selection methodologies are well known to those having ordinary skill in the art. In addition, physical and biochemical methods can be used to identify transformants. These include Southern analysis or PCR amplification for detection of a polynucleotide; Northern blots, S1 RNase protection, primer-extension, quantitative real-time PCR, or reverse transcriptase PCR (RT-PCR) amplification for detecting RNA transcripts; enzymatic assays for detecting enzyme or ribozyme activity of polypeptides and polynucleotides; and protein gel electrophoresis, Western blots, immunoprecipitation, and enzyme-linked immunoassays to detect polypeptides. Other techniques such as in situ hybridization, enzyme staining, and immunostaining also can be used to detect the presence or expression of polypeptides and/or polynucleotides. Methods for performing all of the referenced techniques are well known. After a polynucleotide is stably incorporated into a transgenic plant, it can be introduced into other plants using, for example, standard breeding techniques.

A non-limiting working example of an apple transformation is now presented. Approximately seven to eight weeks prior to transformation using Agrobacterium tumefaciens, apple shoot tip cultures are transferred to a growth medium. Next, at approximately 4-5 weeks prior to transformation, apple shoot tips are transferred to a leaf expansion medium. At approximately one week prior to transformation, appropriate Agrobacterium tumefaciens cultures are prepared according to standard methodologies. Agrobacterium tumefaciens transformation involves harvesting apple leaves and inoculating with the Agrobacterium tumefaciens in M26 cocultivation media. M26 cocultivation media is prepared as follows.

M 26 Cocultivation (1 L)

| | | |
|---|---|---|
| N6 macronutrients | 50 | mL |
| MS micronutrients (10X stock) | 100 | mL |
| MS Vitamin (1000X stock) | 1 | mL |
| NAA | 0.2 | mg/L |
| BAP | 5 | mg/L |
| Gelrite | 2.5 | g/L |
| Sucrose | 30 | g/L |

after autoclaving add:

| | | |
|---|---|---|
| acetosyringone | 1 | mL |
| betaine phosphate | 1 | mL |

Approximately three weeks after inculation, transformed apple plant material is transferred plates to a culture room with no lights. At approximately 4 weeks after transformation light intensity is raised to 10 μE. Regenerants are subsequently identified during a 3-12 week period on a weekly basis. Regenerating meristems are placed in a baby-food jar with M26 Regeneration Medium. M 26 Regeneration Medium (1 L) is prepared as follows.

M 26 Regeneration Medium (1 L)

| | | |
|---|---|---|
| N6 macronutrients | 50 | ml/l |
| MS micronutrients (10X) | 100 | ml/l |
| MS Vitamins (stock 1000X) | 1 | mg/l |
| NAA | 0.2 | mg/l |
| BAP | 5 | mg/l |
| Gelrite | 2.5 | g/l |
| Sorbitol | 30 | g/l |

After autoclaving add Cf 350 (cefotaximine antibiotic). For Cf 350 stock, add 7.1 ml of sterile water to bottle and mix well. Add 1.4 ml/L of this stock to medium.

Approximately 4 weeks after transfer of regenerants to M26 Regeneration medium, meristems are evaluated for transformation as follows. 250-300 randomly chosen regenerants are harvested from leaf-piece transformation plates for each transformation experiment. These regenerants are then tested by PCR for the presence of the transgenes. Here forward and reverse primers based on a sequence of a gene or a construct used for the transformation experiment are employed. Regenerants including the transgene of interest are then confirmed by Southern analysis or any other appropriate method.

Transgenic apple plants (or apple plant cells) can have an altered phenotype as compared to a corresponding control plant (or plant cell) that either lacks the transgene or does not express the transgene. A polypeptide can affect the phenotype of an apple plant (e.g., a transgenic apple plant) when expressed in the apple plant, e.g., at the appropriate time(s), in the appropriate tissue(s), or at the appropriate expression levels. Phenotypic effects can be evaluated relative to a control apple plant that does not express the exogenous polynucleotide of interest, such as a corresponding wild-type plant, a corresponding plant that is not transgenic for the exogenous polynucleotide of interest but otherwise is of the same genetic background as the transgenic plant of interest, or a corresponding plant of the same genetic background in which expression of the polypeptide is suppressed, inhibited, or not induced (e.g., where expression is under the control of an inducible promoter).

### Products

Also provided are compositions such as apple food products and apple feed products, produced using any of the transgenic apple plants described herein.

The transgenic apple plants described herein can be used, for example, to grow apple plants for apple fruit production, or can be used to make apple-based food products such as apple sauce or apple slices, or to produce insoluble fibers. Transgenic apple plants described herein can be used as is or can be used to make food products such as fresh, canned, and frozen fruits.

Transgenic apple plants described herein can also serve as raw materials suitable for fermentation to produce ethanol. For example, apples described herein can be fermented to produce ethyl alcohol. Producing ethanol from plant materials containing increased amounts of sugar can improve ethanol yields.

Transgenic apple plants described herein can also be used as a source from which to extract sugars, using techniques known in the art. Purified sugar, sugar syrup, sugar juice, or extracts containing sugar can be included in nutritional supplements as well as processed food products, e.g., soft drinks, sports drinks, ice cream, baked goods, relishes, sauces, pastes, canned foods, meats, salads, candy, fruit juices, vegetable juices, syrup, snack products, frozen entrees, breakfast cereals, breakfast bars, and baby foods. Sugar can also be included in cell culture media.

Beverages, alcoholic or non-alcoholic, such as apple cider, apple juice, hard cider, apple jack, and apple wine are also produced using the transgenic apples described herein.

### EXAMPLES

The following examples further illustrate the present invention. They are in no way to be construed as a limitation in scope and meaning of the claims.

Apple Transformation without any Selectable Marker

For demonstrating the feasibility of apple transformation without any selectable marker, two binary vector constructs pPin2 mpNPR1 andpwiATT-35SGus containing no nptII selectable marker gene were created. Transgenic apple was obtained using standard methods of Agrobacterium mediated transformation. All the regenerants growing on the media without any selection agent were transferred to individual tubes and were subsequently tested by PCR for the presence of the gene of interest. Two genotypes of apple (M.26 and Galaxy) were chosen to evaluate transformation without a selectable marker.

Materials and Methods

Fully expanded leaves were excised from in vitro growing shoots of Galaxy or M.26 three weeks after subculture. Transformation experiments were carried out as previously described (Borejsza-Wysocka et al., 1999; Norelli et al., 1999) with Agrobacterium tumefaciens strain EHA 105 carrying the binary vector pwiatt-35SGus kan- and pPin2 MpKPR1 kan—(plasmid without the nptII gene). Integration of the attacin E gene and MpNPRI gene was determined by PCR using specific oligonucleotides after DNA extraction from leaves. GUS activity was determined by GUS staining (Jefferson et al, 1987). Ploidy level was checked by flow cytometry as previously described (Reynoird et al., 1999).

Total RNA (50-100 μg) was extracted from 0.5 g of young leaves excised from in vitro shoots as described by Venisse et al. (2002). Reverse transcription (RT) was performed with 2 μg of total RNA. In order to evaluate relative differences in cDNAs between transgenic clones, comparative kinetic analysis was conducted by PCR as suggested by Horikoshi et al. (1992). Initial amounts of PCR substrates were adjusted for each clone on the basis of an equivalent amplification of a cDNA encoding the alpha subunit of translation elongation factor 1 (EF1-α), a member of a constitutively expressed gene family (Mahe et al., 1992). In order to evaluate differences among clones proportional to differences in initial amounts, we limited the amplification to 20 cycles. EF1-α PCR was carried out with the specific primers for apple (forward, 5′-ATTGTGGTCATTGGTCATGT-3′ (SEQ ID NO: 1), and reverse, 5′-CCAATCTTGTAGACATCCTG-3′ (SEQ ID NO:2)). In a second step, comparative amplification was carried out with MpNPR1, pin2, attacin E, Gus and nptII specific primers on equivalent total cDNA. The PCR reaction was run with using standard products from Promega® (Madison, Wis.). The thermocycler program was 94° C., 5 mn, (20 cycles of 94° C., 30 sec, 54° C., 1 min, 72° C., 1 min); 72° C., 15 min for each amplification. After electrophoresis on 1.2% agarose gel, amplified products were blotted onto a Hybond™-N nylon membrane. This membrane was hybridized with a fluorescein labeled probe following the standard procedure of ‘random prime labeling and detection systems’ (Amersham, Piscataway, N.J.).

Results

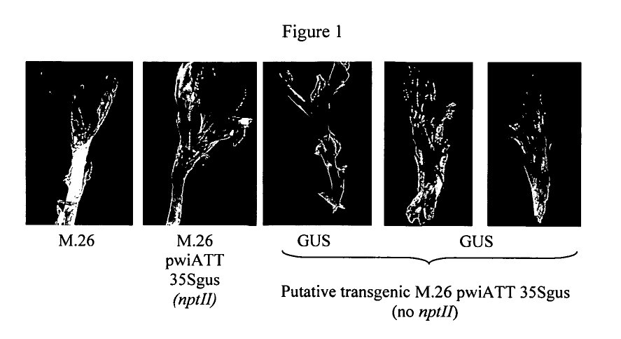

An experiment to transform M.26 with the binary vector pwitAtt 35SGus was performed and 1500 regenerants were harvested from leaf-piece regeneration plates. 566 regenerants were chosen randomly and tested by Gus staining for the presence of the GUS gene. One hundred forty five (25.5%) of them were positive for the presence of GUS (FIG. 1). The expression of the attacin E and Gus genes were confirmed by RT-PCR in selected transgenic lines (data not shown). As expected, no expression of the nptII kanamycin resistance gene was detected in these lines.

Two transformations of the apple rootstock M.26 with the binary vector pPin2MmpNPR1 (no nptII gene) were also performed. 1000 regenerants for each transformation were harvested from leaf-piece transformation plates. 300 and 250 of the regenerants were chosen randomly and tested by PCR for the presence of the pin2 promoter (data not shown). Seventy-six (25.4%) and 55 (22%) of these regenerants, respectively, showed integration of pin2. The parent M.26 was negative for pin2.

Data concerning the transformation of the genotype Galaxy with the binary vector pin2 mpNPR1 or pwiatt35SGus (no nptII gene) was also obtained. 1300 regenerants from each transformation experiment were harvested from leaf-piece transformation plates. Between 150 and 250 of these regenerant were tested by PCR for the presence of the NPR1 gene or tested by Gus staining for the presence of the GUS gene. Eleven and 15 percent of these clones, respectively, showed the integration of the gene.

These aforementioned results showed that we were able to develop a technique for apple transformation without using a selectable marker. This procedure was applied with success to two different genotypes of apple, Galaxy and M.26, with efficiencies of transformation of 12 and 25%, respectively. The impact of this technique is not only its direct utility, but that the end-result, transgenics without marker genes, is so preferable to transgenics with a marker gene (especially kanamycin-resistance), that users, and regulatory agencies, may request in future that approved transgenics be free of marker genes.

In summary, these data provide evidence that markerless transformation of apple can be done in a relatively efficient fashion. The efficiency seen so far is certainly high enough to use on a commercial scale for engineering apple varieties and rootstocks for various traits.

All publications and patent applications mentioned in this specification are indicative of the level of skill of those skilled in the art to which this invention pertains. All publications and patent applications are herein incorporated by reference to the same extent as if each individual publication or patent application was specifically and individually indicated to be incorporated by reference.

Although the invention has been described in detail for the purpose of illustration, it is understood that such detail is solely for that purpose, and variations can be made therein by those skilled in the art without departing form the spirit and scope of the invention which is defined by the following claims.

### REFERENCES

Borejsza-Wysocka E., Norelli J. L., Ko K. and Aldwinckle H. S. 1999. Transformation of authentic M.26 apple rootstock for enhanced resistance to fire blight. Acta Hort. 489: 259-266.
Endo S., Kasahara T., Sugita K., Matsunaga E. and Ebinuma H. 2001. The isopentyl transferase gene is effective as a selectable marker gene for plant transformation in tobacco (Nicotiana tobacum cv. Petite Havana SR1). Plant Cell Rep. 20:60-66.
Haldnip A., Petersen S. G. and Okkels F. T. 1998a. positive selection: a plant selection principle based on xylose isomerase, an enzyme used in food industry. Plant Cell Rep. 18:76-81.
Haldrup A., Petersen S. G. and Okkels F. T. 1998b. The xylose isomerase gene from Thermoanaerobacterium thermosulfurogenes allows effective selection of transgenic plant cells using D-xylose as the selection agent. Plant Mol. Biol. 37:287-296.
Horikoshi T., Danenberg K. D., Stadlauer T. H. W., Volkenandt M., Shea L. C. C., Aigner K., Gustavsson B., Leichman L., Frösing R., Ray M., Gibson N. W., Spears C. P. and Danenberg P. V. 1992. Quantification of thymidylate synthase, dihydrofolate reductase, and DT-diaphorase gene expression in human tumors using the polymerase chain reaction. Cancer Res. 52: 108-116.
Joersbo M., Danaldson I., Kreiberg J., Petersen S. G. and Brunstedt J. 1998. Analasys of mannose selection used for transformation of sugar beet. Mol. Breeding 4:111-117.
Joersbo M. and Okkels F. T. 1996. A novel principle for selection of transgenic plants cells: positive selection. Plant Cell rep. 16:219-221.
Mahe A., Grisvard J. and Dron M. 1992. Fungal and specific gene markers to follow the bean-anthracnose infection process and mormalize the bean chitinase mRNA induction. Mol. Plant Microbe Interact 5: 242-248.
Miki B., and McHugh S. 2004. Selectable marker genes in transgenic plants: applications, alternatives and biosafety; Journal of Biotechnology 107:193-232.
Norelli J. L, Mills J. Z., Momol M. T. and Aldwinckle H. S. 1999. Effect of cecropin-like transgenes on fire blight resistance of apple. Acta Hort. 489: 273-278.
Reynoird J. P., Mourgues F., Norelli J. L; Aldwinckle H. S., Brisset M. N., Chevreau E. 1999. First evidence for improved resistance to fire blight in transgenic pear expressing the attacin E gene from Hyalophora cecropia. Plant Sci. 149: 13-22.
Venisse J. S., Malnoy M., Faize, M., Paulin J. P., Brisset M. N. 2002. Modulation of defense responses of Malus spp. during compatible and incompatible interactions with Erwinia arnylovora. Mol. Plant Microbe Interact. 15: 1204-1212.

What is claimed is:

1. An apple plant or plant component comprising an isolated polynucleotide free from a selectable marker polynucleotide.

2. The apple plant or plant component of claim 1, wherein said selectable marker polynucleotide encodes an herbicide tolerance protein.

3. The apple plant or plant component of claim 1, wherein said selectable marker polynucleotide confers antibiotic resistance.

4. The apple plant or plant component of claim 1, wherein said isolated polynucleotide encodes a disease resistance gene

5. The apple plant or plant component of claim 1, wherein said plant component is apple fruit.

6. The apple plant or plant component of claim 1, wherein said plant component is apple seed.

7. The apple plant or plant component of claim 1, wherein said plant component is apple fruiting scion.

8. The apple plant or plant component of claim 1, wherein said plant component is apple rootstock.

9. The apple plant or plant component of claim 1, wherein said plant component is apple interstem.

10. The apple plant or plant component of claim 1, wherein said plant component is apple pollen.

11. The apple plant or plant component of claim 1, wherein said plant component is apple regenerable tissue.

12. A beverage produced using an apple according to claim 5.

13. A food product produced using an apple according to claim 5.

14. Dietary fiber produced using an apple according to claim 5.

15. A method of transforming an apple plant cell or apple plant tissue using an Agrobacterium mediated process comprising the steps of:
a) inoculating said apple plant cell or apple plant tissue with Agrobacterium containing at least one genetic component capable of being transferred to the apple plant cell or apple tissue in an inoculation media, said inoculation media being free from a selectable marker; and
b) selecting transformed apple plant cells or apple plant tissue.

16. The method of claim 15, further comprising regenerating a transformed apple plant expressing the genetic component from the selected transformed apple plant cells or apple tissue.

17. The method of claim 15, wherein said selectable marker is an antibiotic resistance gene.

18. The method of claim 15, wherein said selectable marker is an herbicide resistance gene.
